(19) United States
(12) Patent Application Publication
(10) Pub. No.: US 2003/0118512 A1
(43) Pub. Date: Jun. 26, 2003

(54) Volatilization of a drug from an inclusion complex

(76) Inventor: William W. Shen, Stanford, CA (US)

Correspondence Address: Richard E. Eckman, Morrison & Foerster LLP, 755 Page Mill Road, Palo Alto, CA, 94304-1018, US

(21) Appl. No.: 10/280,315

(22) Filed: Oct. 25, 2002

Related U.S. Application Data
(60) Provisional application No. 60/335,049, filed on Oct. 30, 2001.
(60) Provisional application No. 60/371,457, filed on Apr. 9, 2002.

(51) Int. Cl. A61K 31/724; A61L 9/04

(52) U.S. Cl. 424/45; 514/58

(57) ABSTRACT

The present invention relates to aerosols or thermal vapors formed from a drug inclusion complex that are used for inhalation therapy. In a method aspect of the invention, a method of delivering a drug to a mammal through an inhalation route is provided which comprises: heating a composition, wherein the composition comprises a drug inclusion complex, to form a drug aerosol or thermal vapor, which is inhaled by the mammal. In a kit aspect of the invention, a kit for delivering a drug through an inhalation route to a mammal is provided which comprises: a) a drug inclusion complex; and, b) a device that forms a drug aerosol or thermal vapor from the drug inclusion complex, for inhalation by the mammal.

### CROSS-REFERENCE TO RELATED APPLICATION

This application claims priority to U.S. provisional application Serial No. 60/335,049 entitled “Volatilization of Nicotine from an Inclusion Complex,” filed Oct. 30, 2001, William W. Shen, the entire disclosure of which is hereby incorporated by reference. This application further claims priority to U.S. provisional application Serial No. 60/371,457 entitled “Volatilization of a drug from an Inclusion Complex,” filed Apr. 9, 2002, William W. Shen, the entire disclosure of which is hereby incorporated by reference.

### FIELD OF THE INVENTION

The present invention relates to the volatilization of a drug from an inclusion complex. Specifically, it relates to aerosols or thermal vapors formed from a drug inclusion complex that are used for inhalation therapy.

### BACKGROUND OF THE INVENTION

Certain drugs suffer from stability problems that prevent them from being used optimally in inhalation delivery systems. It is an object of this invention to provide a drug form that provides for increased stability, thereby allowing drugs to be better used for inhalation therapy.

### SUMMARY OF THE INVENTION

The present invention provides a method and a kit for delivering a drug to a mammal through an inhalation route.

In a method aspect of the invention, a method of delivering a drug to a mammal through an inhalation route is provided which comprises heating a composition, wherein the composition comprises a drug inclusion complex, to form a drug aerosol or thermal vapor, which is inhaled by the mammal.

Typically, the drug inclusion complex is a complex between a drug and a cyclized polysaccharide.

Typically, where the drug inclusion complex is a complex between a drug and a cyclized polysaccharide, the cyclized polysaccharide is either a cyclodextrin or a cyclodextrin derivative.

Typically, where the drug inclusion complex is a complex between a drug and a cyclodextrin, the cyclodextrin is α-cyclodextrin, β-cyclodextrin or γ-cyclodextrin. Preferably, the cyclodextrin is β-cyclodextrin.

Typically, where the drug inclusion complex is between a drug and a cyclodextrin derivative, the cyclodextrin derivative is trimethyl-β-cyclodextrin, dimethyl-β-cyclodextrin, hydroxyethyl-β-cyclodextrin or hydroxypropyl-β-cyclodextrin.

Typically, the drug aerosol or thermal vapor formed from the drug inclusion complex is greater than 95 percent pure. Preferably, the aerosol or vapor formed from the drug inclusion complex is greater than 97 percent pure. More preferably, the aerosol or vapor formed from the drug inclusion complex is greater than 99 percent, 99.5 percent, 99.9 percent or 99.97 percent pure.

In a kit aspect of the invention, a kit for delivering a drug through an inhalation route to a mammal is provided which comprises: a) a drug inclusion complex; and, b) a device that forms a drug aerosol or thermal vapor from the drug inclusion complex, for inhalation by the mammal.

Typically, the drug inclusion complex contained in the kit is a complex between a drug and a cyclized polysaccharide.

Typically, where the drug inclusion complex is a complex between a drug and a cyclized polysaccharide, the cyclized polysaccharide is either a cyclodextrin or a cyclodextrin derivative.

Typically, where the drug inclusion complex is a complex between a drug and a cyclodextrin, the cyclodextrin is α-cyclodextrin, β-cyclodextrin or γ-cyclodextrin. Preferably, the cyclodextrin is β-cyclodextrin.

Typically, where the drug inclusion complex is between a drug and a cyclodextrin derivative, the cyclodextrin derivative is trimethyl-β-cyclodextrin, dimethyl-β-cyclodextrin, hydroxyethyl-β-cyclodextrin or hydroxypropyl-β-cyclodextrin.

Typically, the device contained in the kit comprises: a) an element for heating the drug inclusion complex to form an aerosol or thermal vapor; and, b) an element permitting the mammal to inhale the aerosol or thermal vapor.

### BRIEF DESCRIPTION OF THE FIGURES

FIG. 1 shows a cross-sectional view of a device used to deliver drug aerosols or thermal vapors formed from a drug inclusion complex.

FIG. 2 shows a graph comparing the volatilization rate of pure nicotine at 175° C. to the volatilization rate of nicotine from a β-cyclodextrin inclusion complex at the same temperature.

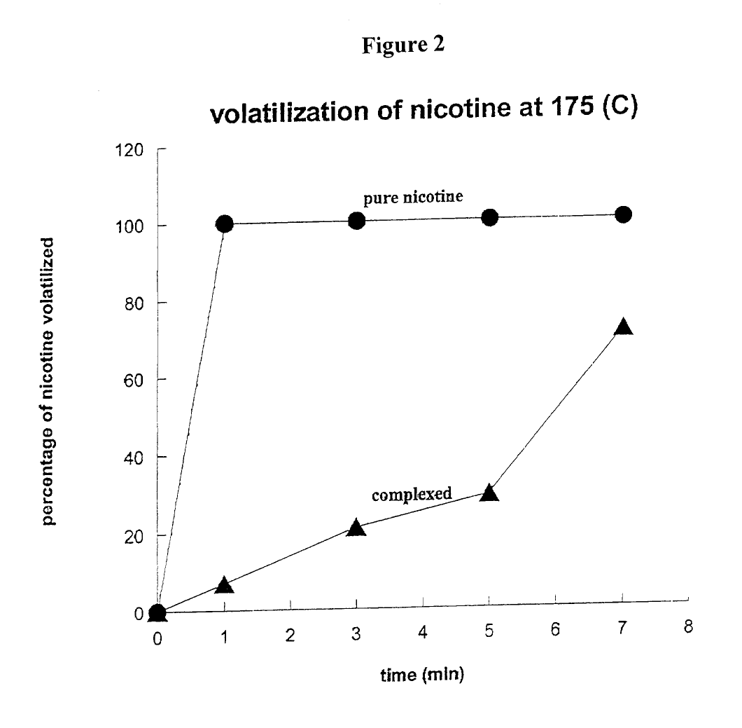

FIG. 3 shows a bar graph comparing the percentage of pure nicotine volatilized at various temperatures for 1 min to the percentage of nicotine volatilized from a β-cyclodextrin inclusion complex at the same temperatures.

### DETAILED DESCRIPTION OF THE INVENTION

Definitions

“Aerosol phase” or “aerosol” refers to solid and/or liquid particles suspended in a gaseous phase.

“Drug” refers to any chemical compound that is used in the prevention, diagnosis, treatment, or cure of disease, for the relief of pain, or to control or improve any physiological or pathological disorder in humans or animals. Classes of drugs include, without limitation, the following: antibiotics, anticonvulsants, antidepressants, antiemetics, antihistamines, antiparkinsonian drugs, antipsychotics, anxiolytics, drugs for erectile dysfunction, drugs for migraine headache, drugs for the treatment of alcoholism, muscle relaxants, nonsteroidal anti-inflammatories, opioids, other analgesics, stimulants and steroids.

Examples of antibiotics include cefmetazole, cefazolin, cephalexin, cefoxitin, cephacetrile, cephaloglycin, cephaloridine, cephalosporin c, cephalotin, cephamycin a, cephamycin b, cephamycin c, cepharin, cephradine, ampicillin, amoxicillin, hetacillin, carfecillin, carindacillin, carbenicillin, amylpenicillin, azidocillin, benzylpenicillin, clometocillin, cloxacillin, cyclacillin, methicillin, nafcillin, 2-pentenylpenicillin, penicillin n, penicillin o, penicillin s, penicillin v, chlorobutin penicillin, dicloxacillin, diphenicillin, heptylpenicillin, and metampicillin.

Examples of anticonvulsants include 4-amino-3-hydroxybutyric acid, ethanedisulfonate, gabapentin, and vigabatrin.

Examples of antidepressants include amitriptyline, amoxapine, benmoxine, butriptyline, clomipramine, desipramine, dosulepin, doxepin, imipramine, kitanserin, lofepramine, medifoxamine, mianserin, maprotoline, mirtazapine, nortriptyline, protriptyline, trimipramine, viloxazine, citalopram, cotinine, duloxetine, fluoxetine, fluvoxamine, milnacipran, nisoxetine, paroxetine, reboxetine, sertraline, tianeptine, acetaphenazine, binedaline, brofaromine, cericlamine, clovoxamine, iproniazid, isocarboxazid, moclobemide, phenyhydrazine, phenelzine, selegiline, sibutramine, tranylcypromine, ademetionine, adrafinil, amesergide, amisulpride, amperozide, benactyzine, bupropion, caroxazone, gepirone, idazoxan, metralindole, milnacipran, minaprine, nefazodone, nomifensine, ritanscrin, roxindole, S-adenosylmethionine, tofenacin, trazodone, tryptophan, venlafaxine, and zalospirone.

Examples of antiemetics include alizapride, azasetron, benzquinamide, bromopride, buclizine, chlorpromazine, cinnarizine, clebopride, cyclizine, diphenhydramine, diphenidol, dolasetron methanesulfonate, droperidol, granisetron, hyoscine, lorazepam, metoclopramide, metopimazine, ondansetron, perphenazine, promethazine, prochlorperazine, scopolamine, tetrahydrocannabinol (THC), triethylperazine, trifluoperazine, triflupromazine, trimethobenzamide, tropisetron, domeridone, and palonosetron.

Examples of antihistamines include azatadine, brompheniramine, chlorpheniramine, clemastine, cyproheptadine, dexmedetomidine, diphenhydramine, doxylamine, hydroxyzine, cetrizine, fexofenadine, loratidine, and promethazine.

Examples of antiparkinsonian drugs include amantadine, baclofen, biperiden, benztropine, orphenadrine, procyclidine, trihexyphenidyl, levodopa, carbidopa, selegiline, deprenyl, andropinirole, apomorphine, benserazide, bromocriptine, budipine, cabergoline, dihydroergokryptine, eliprodil, eptastigmine, ergoline pramipexole, galanthamine, lazabemide, lisuride, mazindol, memantine, mofegiline, pergolike, pramipexole, propentofylline, rasagiline, remacemide, spheramine, terguride, entacapone, and tolcapone.

Examples of antipsychotics include acetophenazine, alizapride, amisulpride, amperozide, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, melperone, mesoridazine, metofenazate, molindone, olanzapine, penfluridol, pericyazine, perphenazine, pimozide, pipamerone, piperacetazine, pipotiazine, prochlorperazine, promazine, quetiapine, remoxipride, risperidone, sertindole, spiperone, sulpiride, thioridazine, thiothixene, trifluperidol, triflupromazine, trifluoperazine, ziprasidone, zotepine, and zuclopenthixol.

Examples of anxiolytics include mecloqualone, medetomidine, metomidate, adinazolam, chlordiazepoxide, clobenzepam, flurazepam, lorazepam, loprazolam, midazolam, alpidem, alseroxlon, amphenidone, azacyclonol, bromisovalum, buspirone, calcium N-carboamoylaspartate, captodiamine, capuride, carbcloral, carbromal, chloral betaine, enciprazine, flesinoxan, ipsapiraone, lesopitron, loxapine, methaqualone, methprylon, propanolol, tandospirone, trazadone, zopiclone, and zolpidem.

Examples of drugs for erectile dysfunction include cialis (IC351), sildenafil, vardenafil, apomorphine, phentolamine, and yohimbine.

Examples of drugs for migraine headaches include almotriptan, alperopride, codeine, dihydroergotamine, ergotamine, eletriptan, frovatriptan, isometheptene, lidocaine, lisuride, metoclopramide, naratriptan, oxycodone, propoxyphene, rizatriptan, sumatriptan, tolfenamic acid, zolmitriptan, amitriptyline, atenolol, clonidine, cyproheptadine, diltiazem, doxepin, fluoxetine, lisinopril, methysergide, metoprolol, nadolol, nortriptyline, paroxetine, pizotifen, pizotyline, propanolol, protriptyline, sertraline, timolol, and verapamil.

Examples of drugs for the treatment of alcoholism include acamprosate, naloxone, naltrexone, and disulfiram.

Examples of muscle relaxants include baclofen, cyclobenzaprine, orphenadrine, quinine, and tizanidine.

Examples of nonsteroidal anti-inflammatories include aceclofenac, alclofenac, alminoprofen, amfenac, aminopropylon, amixetrine, aspirin, benoxaprofen, bermoprofen, bromfenac, bufexamac, butibufen, bucloxate, carprofen, choline, cinchophen, cinmetacin, clidanac, clopriac, clometacin, diclofenac, diflunisal, etodolac, fenclozate, fenoprofen, flutiazin, flurbiprofen, ibuprofen, ibufenac, indomethacin, indoprofen, ketoprofen, ketorolac, loxoprofen, mazipredone, meclofenamate, naproxen, oxaprozin, piroxicam, pirprofen, prodolic acid, salicylate, salsalate, sulindac, tofenamate, and tolmetin.

Examples of opioids include alfentanil, allylprodine, alphaprodine, anileridine, benzylmorphine, bezitramide, buprenorphine, butorphanol, carbiphene, cipramadol, clonitazene, codeine, dextromoramide, dextropropoxyphene, diamorphine, dihydrocodeine, diphenoxylate, dipipanone, fentanyl, hydromorphone, L-alpha acetyl methadol, lofentanil, levorphanol, meperidine, methadone, meptazinol, metopon, morphine, nalbuphine, nalorphine, oxycodone, papaveretum, pethidine, pentazocine, phenazocine, remifentanil, sufentanil, and tramadol.

Examples of other analgesics include apazone, benzpiperylon, benzydramine, bumadizon, clometacin, clonixin, ethoheptazine, flupirtine, nefopam, orphenadrine, propacetamol, and propoxyphene.

Examples of stimulants include amphetamine, brucine, dexfenfluramine, dextroamphetamine, ephedrine, fenfluramine, mazindol, methyphenidate, nicotine, pemoline, phentermine, and sibutramine.

Examples of steroids include betamethasone, chloroprednisone, clocortolone, cortisone, desonide, dexamethasone, desoximetasone, difluprednate, estradiol, fludrocortisone, flumethasone, flunisolide, fluocortolone, fluprednisolone, hydrocortisone, meprednisone, methylprednisolone, paramethasone, prednisolone, prednisone, pregnan-3-alpha-ol-20-one, testosterone, and triamcinolone.

“Thermal vapor” refers to a vapor phase, aerosol phase or mixture of aerosol-vapor phases typically formed by heating.

“Vapor” refers to a gas.

“Vapor phase” refers to a gas phase.

Drug Inclusion Complexes

A drug inclusion complex is formed when a drug molecule binds within a cavity of a host molecule. The binding is noncovalent and can be the result of, for example, ionic interactions, hydrogen bonding or van der Waals interactions. Any molecule that contains a drug binding cavity and that does not substantially degrade (>10% by weight) when kept at a temperature above 200° C. for 1 minute (preferably 1 second) is a suitable host.

Preferably, the host molecule is a cyclized polysaccharide. More preferably, it is a cyclodextrin or cyclodextrin derivative such as α-cyclodextrin, β-cyclodextrin, γ-cyclodextrin, trimethyl-β-cyclodextrin, dimethyl-β-cyclodextrin, hydroxyethyl-β-cyclodextrin or hydroxypropyl-β-cyclodextrin. It is most preferably β-cyclodextrin.

Forming Drug Inclusion Complexes

Drug inclusion complexes are prepared according to well known methods in the art. Typically, a drug and a host molecule are added to water. The mixture/solution is stirred for a few minutes to several hours allowing complexation to occur. Where the inclusion complex is insoluble in the aqueous mixture, isolation is performed by filtration or centrifugation. A soluble complex is usually precipitated by cooling the mixture or by treating it with a chemical agent that facilitates precipitation. The precipitated material is isolated by filtration or centrifugation.

Formation of Drug Aerosols or Thermal Vapors From the Inclusion Complexes

Drug aerosols or thermal vapors of the present invention are formed by heating the inclusion complex. The temperature increase causes the complex to dissociate, thereby releasing aerosol or thermal vapor which is inhaled.

Typically, the inclusion complex is heated on a solid support. The complex is either placed on or adhered to the support. Heat is then applied either directly to the complex or to the support, which transfers heat to the complex.

A number of different materials are used to construct the solid supports. Classes of such materials include, without limitation, metals, inorganic materials, carbonaceous materials and polymers. The following are examples of the material classes: aluminum, silver, gold, stainless steel, copper and tungsten; silica, glass, silicon and alumina; graphite, porous carbons, carbon yams and carbon felts; polytetrafluoroethylene and polyethylene glycol. Combinations of materials and coated variants of materials are used as well.

Where aluminum is used as a solid support, aluminum foil is a suitable material. Examples of silica, alumina and silicon based materials include amorphous silica S-5631 (Sigma, St. Louis, Mo.), BCR171 (an alumina of defined surface area greater than 2 m²/g from Aldrich, St. Louis, Mo.) and a silicon wafer as used in the semiconductor industry. Carbon yams and felts are available from American Kynol, Inc., New York, N.Y. Chromatography resins such as octadecycl silane chemically bonded to porous silica are exemplary coated variants of silica.

The heating of the drug inclusion complexes is performed using any suitable method. Examples of methods by which heat can be generated include the following: passage of current through an electrical resistance element; absorption of electromagnetic radiation, such as microwave or laser light; and, exothermic chemical reactions, such as exothermic salvation, hydration of pyrophoric materials and oxidation of combustible materials.

Delivery of Drug Aerosols or Thermal Vapors

Drug aerosols or thermal vapors of the present invention are delivered to a mammal using an inhalation device. The device has at least two elements: a heating element; and, an element permitting the mammal to inhale the thermal vapor. Various suitable heating methods are described above. The element permitting inhalation is an thermal vapor exit portal that forms a connection to the mammal's respiratory system.

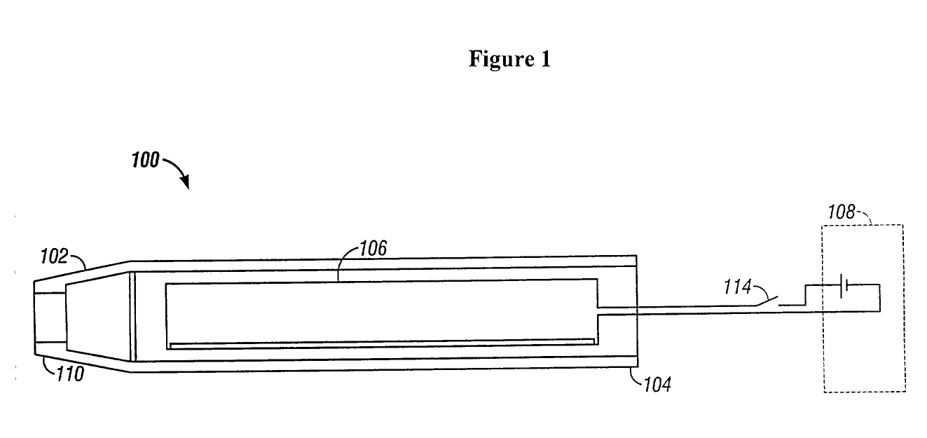

One device used to deliver the drug aerosol or thermal vapor is described in reference to FIG. 1. Delivery device 100 has a proximal end 102 and a distal end 104, a heating module 106, a power source 108, and a mouthpiece 110. Drug inclusion complexes are deposited on a surface 112 of heating module 106. Upon activation of a user activated switch 114, power source 108 initiates heating of heating module 106 (e.g, through ignition of combustible fuel or passage of current through a resistive heating element). The drug volatilizes due to the heating of heating module 106 to form an aerosol or thermal vapor. Air flow travelling from the device distal end 104 to the mouthpiece 110 carries the aerosol or thermal vapor to the mouthpiece 110, where it is inhaled by the mammal.

Dosage of Drug Aerosols or Thermal Vapors for Inhalation Therapy

A typical dosage of a drug aerosol or thermal vapor is either administered as a single inhalation or as a series of inhalations taken within an hour or less (dosage equals sum of inhaled amounts). Where the drug is administered as a series of inhalations, a different amount may be delivered in each inhalation. The dosage amount of a drug in aerosol form is generally no greater than twice the standard dose of the drug given orally.

One can determine the appropriate dose of drug containing aerosols or thermal vapors to treat a particular condition using methods such as animal experiments and a dose-finding (Phase I/II) clinical trial. One animal experiment involves measuring plasma concentrations of an animal after its exposure to the aerosol. Mammals such as dogs or primates are typically used in such studies, since their respiratory systems are similar to that of a human. Initial dose levels for testing in humans is generally less than or equal to the dose in the mammal model that resulted in plasma drug levels associated with a therapeutic effect in humans. Dose escalation in humans is then performed, until either an optimal therapeutic response is obtained or a dose-limiting toxicity is encountered.

The following examples are meant to illustrate, rather than limit, the present invention.

### EXAMPLE 1

### Formation of a Nicotine/β-Cyclodextrin Inclusion Complex

Deionized water (50 mL) was added to β-cyclodextrin (5 g) in a 3-neck, round bottom flask. A condenser was added to the flask, which was then heated to 70° C. in an oil bath. After the β-cyclodextrin dissolved, 0.7 mL of nicotine was added to the flask. The resulting solution was stirred for 4 h and cooled in an ice bath, which induced the precipitation of the nicotine/β-cyclodextrin complex. Precipitate plus solution was transferred to 4 centrifuge tubes, and centrifugation was carried out at 1500 rpm for 5 min. The supernatant was decanted off, and the precipitate was washed with 3 mL of cold, deionized water. The precipitate was dried in an oven for 4 h at 35° C.

### EXAMPLE 2

### Volatilization of Nicotine from a β-Cyclodextrin Inclusion Complex

Nicotine/β-cyclodextrin complex (0.1 g) was sprinkled onto approximately 5 cm² of aluminum foil. The coated aluminum foil was heated, and the evolved vapors were collected as follows: a) a glass tube of 1 inch diameter was pre-heated in a tube furnace to 150° C., 175° C., 200° C. or 250° C.; b) approximately 1 g of glass wool was placed in one end of the glass furnace tube to serve as a trap; c) the trap end of the glass tube was connected to a bubbler containing −70° C. acetone to serve as an additional trap; d) the bubbler was connected to a vacuum pump with an air flow rate of 2 L/min; and, e) the coated aluminum foil was inserted into the center of the furnace tube, and nicotine was volatilized for 60 seconds, with the vaporized nicotine drawn into the various traps by the airflow.

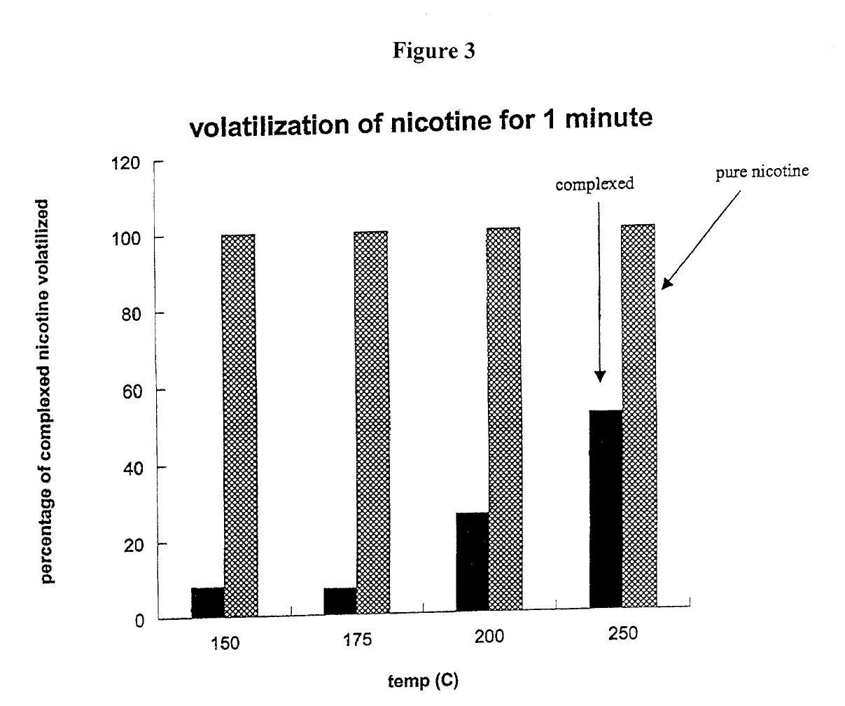

To determine the amount of nicotine volatilized, the aluminum foil, glass tube and glass wool were first extracted with organic solvent (e.g., dichloromethane, acetone or acetonitrile). The extracts, as well as the contents of the acetone bubbler trap, were analyzed by high performance liquid chromatography (HPLC) with a photodiode array detector and/or gas chromatography with a mass spectrometric detector. As shown in FIG. 3, heating the complex for 60 s at different temperatures resulting in different quantities of volatilization: 150° C., 8%; 175° C., 7%; 200° C., 26%; and, 250° C., 52%.

### EXAMPLE 3

### Purity of Nicotine Volatilized from a β-Cyclodextrin Inclusion Complex

Nicotine was volatilized from a nicotine/β-cyclodextrin inclusion complex at 200° C. as described in Example 2. HPLC analysis of the volatilized nicotine showed it to be greater than 99.9% pure.

1. A method of delivering a drug to a mammal through an inhalation device, wherein the method comprises heating a composition to form a drug aerosol or thermal vapor, which is inhaled by the mammal, and wherein the composition comprises a drug inclusion complex.

2. The method of claim 1, wherein the drug inclusion complex is a complex between a drug and a cyclized polysaccharide.

3. The method of claim 2, wherein the cyclized polysaccharide is a cyclodextrin or a cyclodextrin derivative.

4. The method of claim 3, wherein the cyclized polysaccharide is a cyclodextrin, and wherein the cyclodextrin is selected from a group consisting of α-cyclodextrin, β-cyclodextrin, and γ-cyclodextrin.

5. The method of claim 3, wherein the cyclized polysaccharide is a cyclodextrin derivative, and wherein the cyclodextrin derivative is selected from a group consisting of trimethyl-β-cyclodextrin, dimethyl-β-cyclodextrin, hydroxyethyl-β-cyclodextrin and hydroxypropyl-β-cyclodextrin.

6. The method of claim 4, wherein the drug aerosol or thermal vapor formed from the drug inclusion complex is greater than 95 percent pure.

7. The method of claim 5, wherein the drug aerosol or thermal vapor formed from the drug inclusion complex is greater than 95 percent pure.

8. A kit for delivering a drug through an inhalation route to a mammal, wherein the kit comprises:
a) a drug inclusion complex; and,
b) a device that forms a drug aerosol or thermal vapor from the drug inclusion complex
for inhalation by the mammal.

9. A kit according to claim 8, wherein the drug inclusion complex is a complex between a drug and a cyclized polysaccharide.

10. A kit according to claim 8, wherein the device contained in the kit comprises:
a) an element for heating the drug inclusion complex to form an aerosol or thermal vapor; and,
b) an element permitting the mammal to inhale the aerosol or thermal vapor.
